(19) United States
(12) Patent Application Publication
(10) Pub. No.: US 2005/0080470 A1
(43) Pub. Date: Apr. 14, 2005

(54) Intramyocardial lead implantation system and method

(76) Inventors: Randy Westlund, Minneapolis, MN (US); Greg Ley, Blaine, MN (US)

Correspondence Address: Crawford Maunu PLLC;Suite 390, 1270 Northland Drive, St. Paul, MN, 55120, US

(21) Appl. No.: 10/682,371

(22) Filed: Oct. 9, 2003

(51) Int. Cl. A61N 1/05

(52) U.S. Cl. 607/119

(57) ABSTRACT

A system and method for placement of cardiac monitoring and stimulation leads. A lead is advanced into a myocardium of the patient's heart through a lead introducing system, and an electrode is implanted within the myocardium. The method may include gaining access with a cannula from an outer chest wall to the myocardium. Space is created in the myocardium using the lead introduction system. Aspects include eluting a steroid from the lead and into the myocardium, affixing the lead into myocardial tissue using an active fixation mechanism, such as a helical coil, and stabilizing the lead by suturing, clipping, stapling, or other method. A dilating sheath may be used to create a space within the myocardium sufficient in size to accommodate an implantable myocardial electrode. The system may include an implantation depth indicator such as a shoulder, a cuff, or a skirt on the lead.

### FIELD OF THE INVENTION

The present invention relates generally to leads for implantable cardiac monitoring and stimulation devices, and, more particularly, to intramyocardial electrode delivery systems and methods.

### BACKGROUND OF THE INVENTION

Rhythmic contractions of a healthy heart are normally controlled by the sinoatrial (SA) node that includes specialized cells located in the superior right atrium. The SA node is the normal pacemaker of the heart, typically initiating 60-100 heartbeats per minute. When the SA node is pacing the heart normally, the heart is said to be in normal sinus rhythm (NSR).

The heart has specialized conduction pathways in both the atria and the ventricles that enable the rapid conduction of excitation impulses (i.e. depolarizations) from the SA node throughout the myocardium. These specialized conduction pathways conduct the depolarizations from the SA node to the atrial myocardium, to the atrio-ventricular node, and to the ventricular myocardium to produce a coordinated contraction of both atria and both ventricles.

The conduction pathways synchronize the contractions of the muscle fibers of each chamber as well as the contraction of each atrium or ventricle with the contralateral atrium or ventricle. Without the synchronization afforded by the normally functioning specialized conduction pathways, the heart's pumping efficiency is greatly diminished. Patients who exhibit pathology of these conduction pathways can suffer compromised cardiac output, such as that associated with congestive heart failure, for example.

Cardiac rhythm management devices have been developed that provide pacing stimulation to one or more heart chambers in an attempt to improve the rhythm and coordination of atrial and/or ventricular contractions. Cardiac rhythm management devices may also incorporate defibrillation circuitry used to treat patients with serious arrhythmias. Cardiac rhythm management devices typically include circuitry to sense signals from the heart and a pulse generator for providing electrical stimulation to the heart. One or more leads are typically delivered transveneoulsy into the heart, and are coupled to electrodes that contact the myocardium for sensing the heart's electrical signals and for delivering stimulation to the heart in accordance with various therapies. Cardiac rhythm management devices may deliver low energy electrical pace pulses timed to assist the heart in producing a contractile rhythm that maintains cardiac pumping efficiency appropriate to meet the metabolic requirements of the patient.

While transvenous lead delivery is appropriate for many patients that experience adverse synchronization conditions, there are a significant number of patients who could benefit from cardiac resynchronization therapy but are not good candidates for transvenous surgical procedures. Many of these patients are considered poor candidates for transvenous lead implantation for various reasons, including inability to locate the coronary sinus, presence of coronary sinus stenosis, inability to catheterize a desired branch vein, instability of the transvenous lead, or unacceptably high pacing threshold, among others.

### SUMMARY OF THE INVENTION

The present invention is directed to systems and methods for implanting cardiac leads for use with cardiac monitoring and/or stimulation devices. Systems and methods of the present invention are further directed to cardiac lead implantation involving non-transvenous lead delivery.

A method in accordance with one embodiment of the present invention involves gaining access to the pericardium of a patient's chest proximate a cardiac chamber. A lead introducing system is advanced through the pericardium and epicardium, and to the myocardium of the patient's heart. A lead is advanced to the myocardium using the lead introducing system. An electrode of the lead is implanted within the myocardium. The lead introducer system is subsequently removed from the patient's chest.

The method according to this embodiment may further involve gaining access to the epicardium using a cannula advanced from an outer chest wall of the patient. Space may be created in the myocardium using the lead introduction system to provide room for electrode implantation within the myocardium.

In a further embodiment, the lead introducing system includes a piercing needle that can be advanced into the myocardium. A dilating sheath can be advanced over the piercing needle. Myocardial space for electrode implantation can be created using the dilating sheath. The dilating sheath can be removed prior to advancing the lead to the myocardium. The method may include eluting a steroid from the lead and into the myocardium.

A system for facilitating lead implantation in accordance with embodiments of the present invention includes a lead having proximal and distal ends and an open lumen defined therebetween. The lead includes at least one electrode situated at a distal end of the lead. A piercing needle having an outer diameter smaller than a diameter of the open lumen of the lead is configured for advancement into a patient's chest and into an epicardium and myocardium of a heart. A dilating sheath having an inner diameter larger than the outer diameter of the piercing needle includes a distal end configured to create a space within the myocardium sufficient in size to accommodate an implantable myocardial electrode. The relative sizes of the outer diameter of the piercing needle and the diameter of the open lumen of the lead facilitates slideable advancement of the lead over the piercing needle upon removal of the dilating sheath from the patient's chest.

The system may further include a suture sleeve arrangement situated at the distal end of the lead proximal the electrode, the suture sleeve arrangement configured for attachment to an epicardial surface via a suture. The lead and/or electrode may include an attachment arrangement comprising one or more of tines, barbs, helices, and adhesives for affixing the electrode to tissue. The system may further include an implantation depth indicator such as a shoulder, a cuff, or a skirt on the lead.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 illustrates an intramyocardial cardiac monitoring and/or stimulation device in accordance with the present invention, as implanted in a patient;

FIG. 2 is an exploded diagram showing components of an intramyocardial lead placement system in accordance with an embodiment of the present invention;

FIG. 3 illustrates a thoracoscopic method of intramyocardial lead implantation in accordance with an embodiment of the present invention;

FIG. 4 illustrates an intramyocardial lead having helical fixation in the myocardium and chronic fixation in the pericardium in accordance with an embodiment of the present invention;

FIG. 5 illustrates an embodiment of an intramyocardial lead in accordance with the present invention; and

FIG. 6 illustrates an embodiment of an intramyocardial lead having a helical fixation element in accordance with the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

Methods and devices employing an implantable lead system in accordance with the present invention may incorporate one or more of the features, structures, methods, or combinations thereof described herein below. For example, an intramyocardial lead implantation system may be implemented to include one or more of the features and/or processes described below. It is intended that such a device or method need not include all of the features and functions described herein, but may be implemented to include one or more selected features and functions that provide for unique structures and/or functionality.

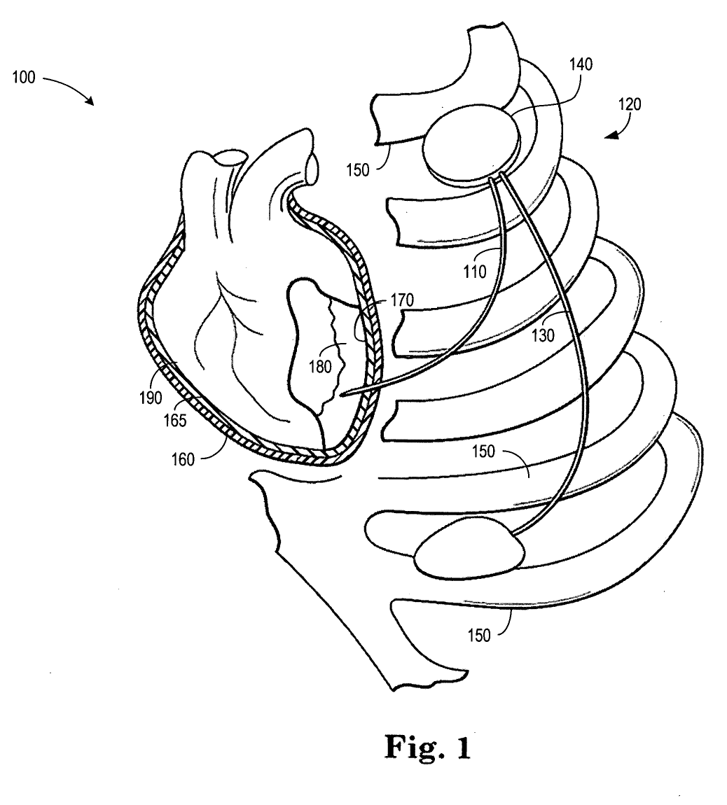

FIG. 1 illustrates an intramyocardial cardiac monitoring and/or stimulation system 100 in accordance with the present invention, as implanted in the heart 190 of a patient. In general terms, an intramyocardial lead 110 implemented in accordance with the present invention may be used with a cardiac monitoring and/or stimulation device. One such device is an implantable cardiac monitoring and/or stimulation (ICMS) device 120 that includes a housing or can 140 implanted under the skin in the chest region of a patient.

The can 140 of the ICMS device 120 may be configured for positioning outside of the rib cage at an intercostal or subcostal location, within the abdomen, or in the upper chest region and include one or more intramyocardial leads 110 having one or more electrodes implanted within myocardial tissue of the heart. Although a single intramyocardial lead 110 is shown implanted in the left ventricle in FIG. 1, it is understood that one or more intramyocardial leads 110 may be implanted in myocardial tissue of one or more chambers of the heart.

The intramyocardial lead 110 shown in FIG. 1 is connected to the can 140 of the ICMS device 120. The can 140 is positioned external of the patient's rib cage 150. The intramyocardial lead 110 extends from the can 140, through the intercostal space, and into the thoracic cavity. The intramyocardial lead 110 penetrates the pericardium 160, epicardium 170, and into the myocardium 180 of the heart 190.

The ICMS device 120 may also be used with other leads, such as, for example, a subcutaneous lead 130. The subcutaneous lead 130 may be used for monitoring and/or stimulation in combination with one or more of the intramyocardial lead(s) 110. For example, subcutaneous leads that may be used in cooperation with the ICMS system 100 are disclosed in commonly owned U.S. patent application Ser. No. 10/465,520, filed Jun. 19, 2003 [Attorney Docket Number GUID.615PA], which is hereby incorporated herein by reference. One or more intramyocardial leads 110 may further be used in combination with a subcutaneous monitoring and/or stimulation device of the type disclosed in commonly owned U.S. patent application Ser. No. 10/462,001, filed Jun. 13, 2003 [Attorney Docket Number GUID.612PA], which is hereby incorporated herein by reference.

The ICMS device 120 shown in FIG. 1 is intended to be representative of various types of cardiac rhythm management devices. Such devices include, for example, implantable pulse generators such as pacemakers and implantable cardioverter/defibrillators that provide electrical stimulation to selected chambers of the heart. A pacemaker, for example, is an implantable pulse generator that paces the heart with timed pacing pulses. The most common condition for which pacemakers are used is in the treatment of bradycardia, where the ventricular rate is too slow. Atrio-ventricular conduction defects (i.e., AV block) that are permanent or intermittent and sick sinus syndrome represent the most common causes of bradycardia for which permanent pacing may be indicated. If functioning properly, the pacemaker makes up for the heart's inability to pace itself at an appropriate rhythm in order to meet metabolic demand by enforcing a minimum heart rate.

Another embodiment of the ICMS device 120 is a cardiac resynchronization device, which monitors and regulates the degree to which the heart chambers contract in a coordinated manner during a cardiac cycle to effect efficient pumping of blood. The heart has specialized conduction pathways in both the atria and the ventricles that enable the rapid conduction of excitation (i.e., depolarization) throughout the myocardium. These pathways conduct excitatory impulses from the sino-atrial node to the atrial myocardium, to the atrio-ventricular node, and thence to the ventricular myocardium to result in a coordinated contraction of both atria and both ventricles. This both synchronizes the contractions of the muscle fibers of each chamber and synchronizes the contraction of each atrium or ventricle with the contralateral atrium or ventricle. Without the synchronization afforded by the normally functioning specialized conduction pathways, the heart's pumping efficiency is greatly diminished. Patients who exhibit pathology of these conduction pathways, such as bundle branch blocks, can thus suffer compromised cardiac output.

Heart failure, for example, is a clinical syndrome in which an abnormality of cardiac function causes cardiac output to fall below a level adequate to meet the metabolic demand of peripheral tissues and is usually referred to as congestive heart failure (CHF) due to the accompanying venous and pulmonary congestion. CHF can be due to a variety of etiologies, with ischemic heart disease being the most common. Some CHF patients suffer from some degree of AV block or are chronotropically deficient such that their cardiac output may be improved with conventional bradycardia pacing. Such pacing, however, may result in some degree of uncoordination in atrial and/or ventricular contractions due to the way in which pacing excitation is spread throughout the myocardium. The resulting diminishment in cardiac output may be significant in a CHF patient whose cardiac output is already compromised. Intraventricular and/or interventricular conduction defects (e.g., bundle branch blocks) are also commonly found in CHF patients.

ICMS device 120 may be configured to treat these problems, such as by providing electrical pacing stimulation to one or both ventricles in an attempt to improve the coordination of ventricular contractions, termed cardiac resynchronization therapy. The ICMS device 120 may be configured structurally and functionally in a manner described in commonly owned U.S. Pat. Nos. 6,597,951; 6,574,506; 6,512,952; 6,501,988; 6,411,848; and 6,363,278, each of which is hereby incorporated herein by reference.

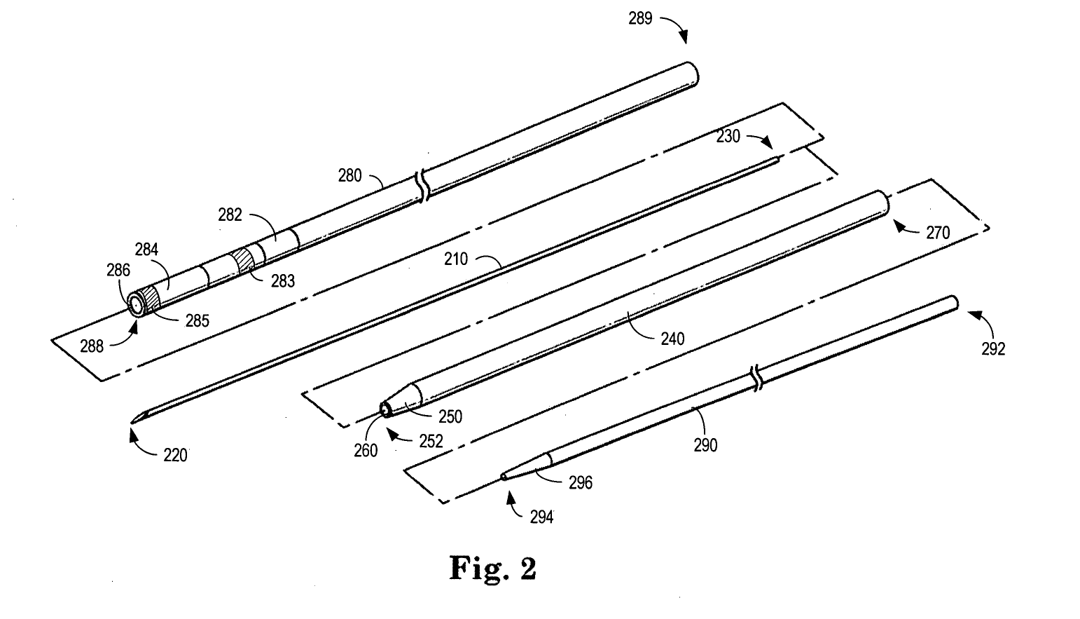

FIG. 2 is a diagram showing various components of an intramyocardial lead placement system in accordance with an embodiment of the present invention. The system, according to this embodiment, includes a piercing needle 210 that has a tip 220 and a proximal end 230. A dilating catheter 240 of the system includes a lumen 260 providing an opening between a distal end 252 and a proximal end 270 of the dilating catheter 240. A dilating portion 250 of the dilating catheter 240 is provided at the proximal end 252. A dilating sheath 290 includes a proximal end 292, a distal end 294, and a dilating portion 296. An open lumen lead 280 includes a lumen 286 providing access between a proximal end 289 and a distal end 288 of the lead 280. The lead 280 may include one or more electrodes, such as, for example, a first electrode 282 and a second electrode 284.

The electrodes 282 and 284 are adapted to provide sensing and/or stimulation to the myocardium of the heart 190 (seen in FIG. 1). The electrodes may be, for example, electrodes manufactured from metals or metal covered polymers, such as those described in commonly owned U.S. Pat. No. 5,554,178, which is hereby incorporated herein by reference.

The distal portion 288 of the lead 280, which defines the electrode region, is preferably very flexible in order to reduce chronic irritation of cardiac tissue. The distal portion 288 may be constructed to include both a flexible insulator, such as a polymer insulator, and a flexible conductor to which the tip electrode 284 is coupled. Suitable conductors may include one or more of, for example, a nitinol coil conductor, a platinum or tantalum clad MP35N coil conductor, a single or multiple filar wound conductor, or a cable conductor. The electrode 282 may be fabricated to be a high micro-surface area electrode, such as an electrode having an iridium oxide micro-surface. The electrode 282 may be mounted circumferentially about the distal tip of the lead 280 or may be mounted to the lead 280 such as, for example, by mounting to the distal end of the lead 280 and extending perpendicularly with respect the longitudinal axis of the lead 280.

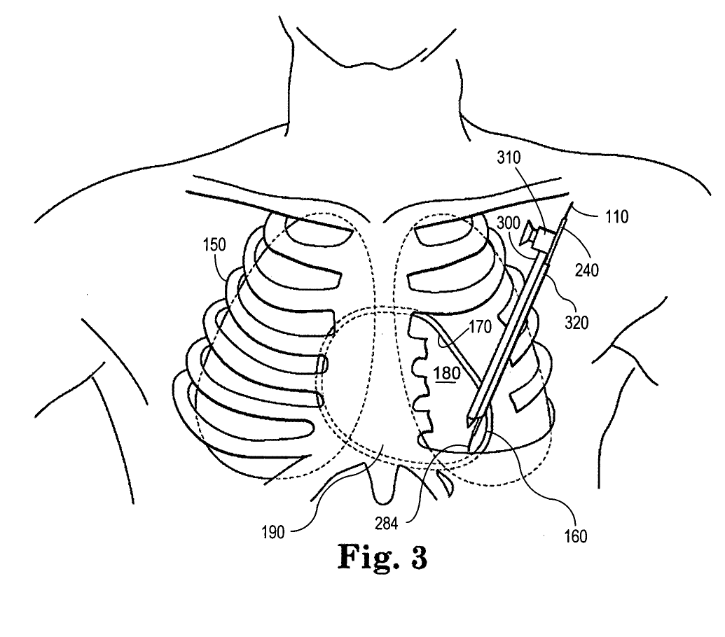

FIG. 3 illustrates a thoracoscopic method of intramyocardial lead implantation in accordance with an embodiment of the present invention. For purposes of the following discussion, reference is made to components of both FIGS. 3 and 2.

A cannula 300, such as, for example, a trocar, is inserted through the chest and intercostal region of the rib cage 150, providing access into the intrathoracic area, and up to the pericardium 160. A thoracoscope 310 may be used to help visualize and direct the procedure. The procedure may also be accomplished using other guidance methods such as, for example, MRI, ultrasound, CT, fluoroscopy, or other visualization method.

The cannula 300 may have a working channel 320 providing access for the lead 110. The piercing needle 210 (see FIG. 2) is passed through the channel 320, and pierces through the pericardium 160 and enters the myocardium 180. An effective depth of penetration of the piercing needle 210 is about 5 mm into the myocardium 180, preferably at a shallow angle substantially tangential to the epicardial surface. Useful angles of insertion are between about 5 degrees and about 45 degrees with respect to a plane tangential to the epicardial surface at the insertion point.

The dilating sheath 290 (shown in FIG. 2) may then be advanced over the piercing needle 210 to create a space or cavity in the myocardium 180. The dilating sheath 290 is removed, and the lead 280 is advanced over the piercing needle 210 and directed to the space created by the dilating sheath 290. The electrode 284 is implanted within the myocardium 180 beneath the epicardium 170. It is noted that creation of the myocardial space is a preferred but optional procedure.

The dilating sheath 290, cannula 300, and dilating catheter 240, are typically sufficiently rigid to provide access and/or penetration capabilities. The dilating sheath 290, cannula 300, and dilating catheter 240 should also be sufficiently flexible to project through the rib cage 150 and extend to the heart 190. The dilating sheath 290, cannula 300, and dilating catheter 240 may be formed from a malleable and/or bendable material such as, for example, metal or polymers capable of deflection or deformation. In another embodiment, the dilating sheath 290, cannula 300, and dilating catheter 240 may be pre-formed from a rigid material such as, for example, structural composite, rigid metals, or stiff polymers. In one configuration, the dilating catheter 240 is configured as a splittable dilating catheter formed from a polymeric material and comprising one or more longitudinal splitting features (e.g., pre-stress lines or perforated lines) defined along a length of the dilating catheter 240.

It may be beneficial to provide a pharmacological eluting element on or proximate the lead/electrode 280/284 to, for example, reduce inflammation, aid in healing, and/or reduce pain. For example, one or more steroid-eluting collars 283, 285 may be provided at the distal end of the lead 280 to reduce inflammation. The steroid-eluting collars 283, 285 may be positioned distal to the electrodes 282, 284, as is shown in FIG. 2. The steroid-eluting collars 283, 285 may also be positioned proximal of the electrodes 282, 284 or both distal to and proximal of the electrodes 282, 284. Leads and collars useful for drug delivery are described in commonly owned U.S. Pat. Nos. 4,819,662 and 6,361,780, which are hereby incorporated herein by reference.

After placement of the electrode 284 within the myocardium 180, the piercing needle 210 and the thoracoscope 310 are removed. It may be useful to provide fixation of the lead 280 with respect to cardiac and/or pericardial tissue, as will be described in more detail below.

Still referring to FIGS. 3 and 2, another embodiment of a method of intramyocardial lead implantation in accordance with the present invention employs the dilating catheter 240. Access and delivery of the piercing needle 210 are performed as described above. After inserting the dilating sheath 290 over the piercing needle 210, the dilating catheter 240 is then inserted over the dilating sheath. The dilating catheter 240 is held in position as both the piercing needle 210 and the dilating sheath 290 are withdrawn. The lumen 260 of the dilating catheter 240 now provides access directly to the myocardium for placement of any desired lead. After lead placement and electrode implantation, the catheter 240 and any other access devices are removed. Before the thoracoscope 310 and cannula 300 are removed, the lead may be fixed at one or more sites to assure efficacy.

The dilating catheter 240 may include perforations or other features facilitating the stripping of the catheter 240 from the lead 280. For example, the catheter 240 may be provided with perforations along the entire length of the catheter 240. After fixing the lead 280 into the myocardium, a finishing wire (not shown) may be inserted into the lumen 286 of the lead 280, holding the lead 280 in place while the catheter 240 is stripped from the lead 280 and pulled from the patient. The finishing wire may then be removed, leaving the lead 280 implanted in the patient.

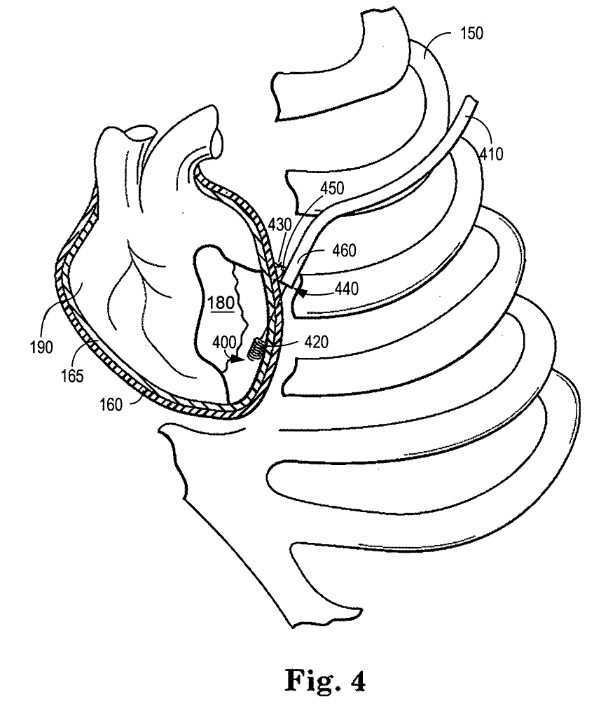

Turning now to FIG. 4, there is illustrated an intramyocardial lead 410 having a helical fixation element 420 implanted in the myocardium 180 in accordance with an embodiment of the present invention. In addition to acute fixation vis-à-vis the helical fixation element 420, the intramyocardial lead 410 may further include an acute fixation arrangement or element 430 positioned in the pericardium 160.

During delivery of the lead 410, the fixation element 420 is implanted within the myocardium 180 by rotating the lead 410. As the lead 410 is rotated, the sharp end 400 of the helical fixation element 420 engages myocardial tissue and penetrates into the myocardium 180. As the lead 410 is further rotated, the sharp end 400 burrows through the tissue, penetrating further into myocardial tissue and acutely fixing the electrode within the myocardium 180. This process effectively screws the helical fixation element 420 into the myocardial tissue.

Although helical fixation element 420 is illustrated having uniform pitch, cylindrical cross-section, and constant coil thickness, it is contemplated that any helical or screw-like structure may be used in accordance with the present invention. The helix may be of non-uniform and/or tapering cross-section, the pitch may be non-uniform, and the shape and thickness of the coil may be varied, for example.

The lead 410 is also illustrated to include a depth indicator providing a reference to the clinician of how far the lead 410 and/or electrode is inserted into the myocardial tissue. The step 440 may be used as an indicator to judge insertion depth into tissue, and/or to limit insertion of the electrode by, for example, stopping the insertion at the pericardium 160, as indicated in FIG. 4. It is to be understood that the step 440 is but one example of a depth indicator, and that any depth indicator may be used in accordance with the present invention such as, for example, a cuff, skirt, shoulder, or other structure or marking that provides for insertion depth feedback.

The helical fixation element 420 may be used in combination with the acute fixation element 430 and/or chronic fixation elements such as, for example adhesion sites 460 further described below, to maintain the electrode and lead placement. The acute fixation element 430 may be, for example, a suture as is illustrated in FIG. 4, or may be any appropriate fixation device such as, for example, a clip, a staple, or other fixation element. The helical fixation element 420 may be a conductor used as an electrode, or may be used only as a fixation element.

The lead 410 may further incorporate a fixation site 450, here illustrated as a loop available for passing a suture needle and suture during acute fixation. The fixation site 450 may be any useful fixation-assisting element such as, for example, a sponge, an aperture, a collar, or other suitable element. The fixation site 450 may additionally, or alternately, include one or more adhesion sites 460 to chronically fix the lead 410.

In accordance with a further configuration, the adhesion sites 460 may include a material that promotes tissue in-growth or attachment at the adhesion sites 460. For example, the bulk outer sleeve of the body of lead 410 may be constructed such that it includes a first polymer material that substantially prevents tissue in-growth. Selective portions of the body of lead 410 may include adhesion sites 460 formed using a second polymer material that promotes tissue in-growth or attachment between the adhesion sites 460 and tissue contacting the adhesion sites 460. The second polymer material may, for example, have a porosity, pore sizes or distribution of pore sizes that differ from that of the first polymer material. By way of further example, the second polymer material may differ in terms of hydrophobicity relative to the first polymer material.

In one particular configuration, the first polymer material may include a first type of PTFE (polytetrafluoroethylene), and the second polymer material of the adhesion sites 460 may include a second type of PTFE. In one particular arrangement, the first type of PTFE includes a first type of ePTFE (expanded polytetrafluoroethylene), and the second type of PTFE includes a second type of ePTFE. The second type of ePTFE preferably differs from the first type of ePTFE in terms of one or more of porosity, pore sizes or distribution of pore sizes. Additional details of fixation approaches involving surface texturing, selective material use, and other arrangements that facilitate lead/electrode fixation via tissue ingrowth are disclosed in commonly owned U.S. patent application Ser. No. 10/004,708 (GUID.031 US01) filed Dec. 4, 2001 and entitled “Apparatus and Method for Stabilizing an Implantable Lead,” which is hereby incorporated herein by reference.

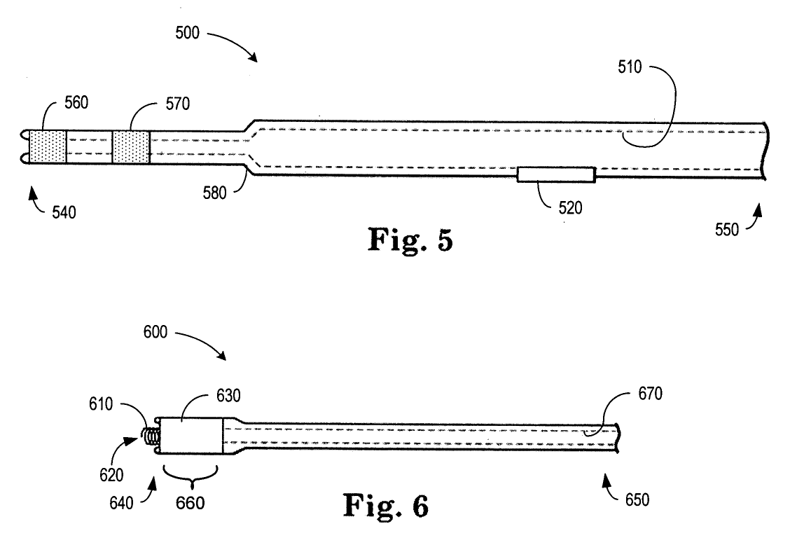

FIGS. 5 and 6 illustrate embodiments of intramyocardial leads in accordance with the present invention. FIG. 5 illustrates an open lumen lead 500 in accordance with the present invention. The lead 500 includes a lumen 510 providing access between a distal end 540 and a proximal end 550. A suture site 520 is located approximately 10 mm proximal of the distal end 540. The suture site 520 may be an easily penetrable portion of the lead 500 providing a convenient and easy site for needle penetration during suturing, or for example, a convenient stapling site or clip location.

A first electrode 560 and a second electrode 570 are illustrated within the first 5 mm of the distal end 540, and distal to a step 580. The electrodes 560 and 570 are illustrated as cylindrical electrodes, having an increased surface area treatment such as, for example, iridium oxide or other suitable coating that increases the surface area of the electrode and improves conduction to myocardial tissue.

The step 580 may be provided as a convenient indicator of the proper insertion depth for the lead 500 into the myocardial tissue. If a particular lead is properly inserted to a depth of 5 mm, for example, the lead may be designed with a step, rule, marking, or other indicator providing the clinician with a reference during the lead 500 insertion.

FIG. 6 illustrates an embodiment of an intramyocardial lead 600 having a helical fixation element 610 in accordance with the present invention. The helical element 610 may be provided with a sharp tip 620 for ease of insertion into myocardial tissue. The lead 600 includes a lumen 670 providing access between a distal end 640 and a proximal end 650. An electrode 630 is illustrated at the distal end 640 of the lead 600. The electrode 630 is illustrated as a cylindrical electrode, and may be electrically coupled to, or insulated from, the helical fixation element 610.

The helical fixation element 610 may constitute an electrode for the system, in which case electrode 620 need not be included. As mentioned above, the helical fixation element 610 may be used only for fixation. The element 610 may also be used in combination with one or more electrodes 630, useful for bi-polar pair electrodes or other applications.

The lead 600 is illustrated with a cuff 660 at the distal end 640. The cuff 660 may be provided as a convenient indicator of the proper insertion depth for the lead 600 into the myocardial tissue. If a particular lead is properly inserted to a depth of 5 mm, for example, the lead may be designed with a cuff 660, skirt, rule, marking, or other indicator at about 5 mm proximal of the distal end for providing the clinician with a reference during the lead 600 insertion.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

1. A minimally invasive method of implanting a lead, comprising:
gaining access to a pericardium of a patient's chest proximate a cardiac chamber;
advancing a lead introducing system through the pericardium and epicardium, and to myocardium of the patient's heart;
advancing a lead to the myocardium using the lead introducing system;
implanting an electrode of the lead within the myocardium; and
removing the lead introducer system from the patient's chest.

2. The method of claim 1, further comprising gaining access to the epicardium using a cannula advanced from an outer chest wall of the patient.

3. The method of claim 1, further comprising creating a space in the myocardium using the lead introduction system.

4. The method of claim 3, wherein advancing the lead introducing system further comprises advancing a piercing needle into the myocardium.

5. The method of claim 3, wherein:
advancing the lead introducing system further comprises:
advancing a piercing needle into the myocardium; and
advancing a dilating sheath over the piercing needle; and
creating the myocardial space further comprises using the dilating sheath to create the space in the myocardium.

6. The method of claim 5, further comprising removing the dilating sheath prior to advancing the lead using the lead introducing system.

7. The method of claim 1, further comprising eluting a steroid from the lead and into the myocardium.

8. The method of claim 1, wherein implanting the electrode comprises affixing the electrode to myocardial tissue.

9. The method of claim 8, wherein affixing the electrode comprises using an active fixation mechanism that affixes the electrode to the myocardial tissue.

10. The method of claim 9, wherein the active fixation mechanism comprises a helical mechanism.

11. The method of claim 10, wherein the helical mechanism is positioned coaxial to a longitudinal axis of the lead.

12. The method of claim 1, wherein implanting the electrode comprises stabilizing the lead proximally of the electrode.

13. The method of claim 12, wherein stabilizing the lead comprises stabilizing the lead via attachment of a portion of the lead to an epicardial surface of the heart.

14. The method of claim 1, wherein implanting the electrode comprises placing a fixation element epicardially at or near a location at which the lead enters the myocardium.

15. The method of claim 14, wherein the fixation element comprises a suture.

16. The method of claim 14, wherein the fixation element comprises a clip.

17. The method of claim 14, wherein the fixation element comprises a chronic fixation arrangement.

18. The method of claim 1, wherein entering the myocardium comprises entering the myocardium at a shallow angle substantially tangential to an epicardial surface and penetrating the myocardium to a predetermined depth.

19. The method of claim 18, wherein the shallow angle ranges between about 5 degrees and about 45 degrees with respect to a plane tangential to the epicardial surface, and the predetermined depth ranges between about 1 mm and about 10 mm.

20. The method of claim 1, wherein the lead is implanted proximate a left ventricle of the heart.

21. The method of claim 1, wherein:
the lead comprises proximal and distal ends and an open lumen defined therebetween;
advancing the lead introducing system further comprises advancing a piercing needle into the myocardium and into the epicardium and myocardium; and
advancing the lead comprises passing the lead over the piercing needle and through the open lumen.

22. The method of claim 21, further comprising engaging an active fixation mechanism that affixes the electrode within the myocardium.

23. The method of claim 21, wherein advancing the lead introducing system further comprises:
advancing a dilating sheath over the piercing needle, the dilating sheath creating a space within the myocardium; and
removing the dilating sheath prior to passing the lead over the piercing needle.

24. The method of claim 1, wherein advancing the lead introducing system further comprises:
advancing a piercing needle into the epicardium and myocardium;
advancing a dilating sheath over the piercing needle, the dilating sheath creating a space within the myocardium;
advancing a dilating catheter over the dilating sheath, wherein advancing the lead comprises passing the lead through the dilating catheter; and
removing the piercing needle and dilating sheath.

25. The method of claim 24, further comprising using a stylet that stiffens the lead to facilitate advancement of the lead through the dilating catheter.

26. The method of claim 24, further comprising engaging an active fixation mechanism that affixes the electrode within the myocardium.

27. A system for facilitating cardiac lead implantation, comprising:
a lead comprising proximal and distal ends and an open lumen defined therebetween, the lead further comprising at least one electrode situated at a distal end of the lead;
a piercing needle having an outer diameter smaller than a diameter of the open lumen of the lead, the piercing needle configured for advancement into a patient's chest and into an epicardium and myocardium of a heart; and
a dilating sheath having an inner diameter larger than the outer diameter of the piercing needle, the dilating sheath having a distal end configured to create a space within the myocardium sufficient in size to accommodate the at least one electrode;
wherein the relative sizes of the outer diameter of the piercing needle and the diameter of the open lumen of the lead facilitates slideable advancement of the lead over the piercing needle upon removal of the dilating sheath from the patient's chest.

28. The system of claim 27, wherein the lead further comprises a suture sleeve arrangement situated at the distal end of the lead proximal the electrode, the suture sleeve arrangement configured for attachment to an epicardial surface via a suture.

29. The system of claim 27, wherein the electrode comprises an attachment arrangement comprising one or more of tines, barbs, helices, and adhesives for affixing the electrode within the myocardium.

30. The system of claim 27, wherein the lead further comprises an implantation depth indicator.

31. The system of claim 30, wherein the implantation depth indicator comprises a shoulder of the lead.

32. The system of claim 30, wherein the implantation depth indicator comprises a cuff of the lead.

33. The system of claim 30, wherein the implantation depth indicator comprises a skirt of the lead.

34. The system of claim 27, wherein the dilating sheath is formed from a polymeric or composite material.

35. The system of claim 34, wherein the dilating sheath is rigid.

36. The system of claim 34, wherein the dilating sheath is flexible.

37. The system of claim 27, wherein the dilating sheath is formed from a metal.

38. The system of claim 37, wherein the dilating sheath is rigid.

39. The system of claim 37, wherein the dilating sheath is flexible.

40. The system of claim 27, wherein the electrode is situated on a distal tip of the lead and substantially perpendicular to a longitudinal axis of the lead.

41. The system of claim 27, wherein the electrode is situated circumferentially at a distal tip of the lead.

42. The system of claim 27, wherein the electrode comprises a micro surface comprising iridium oxide.

43. The system of claim 27, wherein the electrode comprises a steroid eluting arrangement disposed on one or a plurality of sides of the electrode.

44. The system of claim 27, wherein a distal tip of the lead comprises flexible insulation material and a flexible conductor.

45. The system of claim 44, wherein the flexible conductor comprises one or more of a nitinol coil conductor, a platinum clad nitinol coil conductor, a single or multiple filar wound conductor, a stainless steel conductor, an MP35N conductor, a tantalum conductor, a titanium alloy conductor, and a cable conductor.

46. A system for facilitating lead implantation, comprising:
a lead comprising proximal and distal ends and an open lumen defined therebetween, the lead further comprising at least one electrode situated at a distal end of the lead;
a piercing needle having an outer diameter smaller than a diameter of the open lumen of the lead, the piercing needle configured for advancement into a patient's chest and into an epicardium and myocardium of a chamber of a heart;
a dilating sheath having an inner diameter larger than the outer diameter of the piercing needle, the dilating sheath having a distal end configured to create a space within the myocardium sufficient in size to accommodate the at least one electrode; and
a dilating catheter having an inner diameter larger than an outer diameter of the dilating sheath and an outer diameter larger than an outer diameter of the lead;
wherein the relative sizes of the inner diameter of the dilating catheter and outer diameter of the lead facilitates advancement of the lead within the dilating catheter upon removal of the piercing needle and dilating sheath from the patient's chest.

47. The system of claim 46, wherein one or both of the dilating sheath and dilating catheter are formed from a polymeric material.

48. The system of claim 46, wherein the dilating catheter is configured as a splittable dilating catheter formed from a polymeric material and comprising one or more longitudinal splitting features defined along a length of the dilating catheter.

49. The system of claim 46, wherein one or both of the dilating sheath and dilating catheter are formed from a metal.

50. The system of claim 46, wherein the lead further comprises a suture sleeve arrangement situated on the distal end of the lead proximal of the electrode, the suture sleeve arrangement configured for attachment to an epicardial surface via a suture.

51. The system of claim 46, wherein the lead comprises a stylet situated at the distal end of the lead that enhances stiffness of the lead.

52. The system of claim 46, wherein the electrode comprises an electrode attachment arrangement comprising one or more of tines, barbs, and adhesives for affixing the electrode to myocardial tissue.

53. The system of claim 46, wherein the electrode comprises an active fixation mechanism for affixing the electrode within the myocardium.

54. The system of claim 46, wherein the electrode comprises one or more helices for affixing the electrode within the myocardium.

55. The system of claim 54, wherein the one or more helices comprise extendible/retractable fixation helices for affixing the electrode within the myocardium.

56. The system of claim 55, wherein the one or more helices are positioned coaxial to a longitudinal axis of the lead.

57. The system of claim 46, wherein the electrode is situated on a distal tip of the lead and substantially perpendicular to a longitudinal axis of the lead.

58. The system of claim 46, wherein the electrode is situated circumferentially about a distal tip of the lead.

59. The system of claim 46, wherein the electrode comprises a micro surface comprising iridium oxide.

60. The system of claim 46, wherein the electrode comprises a steroid eluting arrangement disposed on one or a plurality of sides of the electrode.

61. The system of claim 46, wherein a distal tip of the lead comprises flexible insulation material and a flexible conductor.

62. The system of claim 61, wherein the flexible conductor comprises one or more of a nitinol coil conductor, a platinum clad nitinol coil conductor, a single or multiple filar wound conductor, a stainless steel conductor, an MP35N conductor, a tantalum conductor, a titanium alloy conductor, and a cable conductor.

63. The system of claim 46, wherein the lead further comprises an implantation depth indicator.

64. The system of claim 63, wherein the implantation depth indicator comprises a shoulder of the lead.

65. The system of claim 63, wherein the implantation depth indicator comprises a cuff of the lead.

66. The system of claim 63, wherein the implantation depth indicator comprises a skirt of the lead.
